(19) United States
(12) Patent Application Publication
(10) Pub. No.: US 2021/0090686 A1
(43) Pub. Date: Mar. 25, 2021

(54) SINGLE CELL RNA-SEQ DATA PROCESSING

(71) Applicant: Regeneron Pharmaceuticals, Inc., Tarrytown, NY (US)

(72) Inventors: Gurinder Singh Atwal, New York, NY (US); Wei Keat Lim, Scarsdale, NY (US); Ruoyu Zhang, White Plains, NY (US)

(21) Appl. No.: 17/032,848

(22) Filed: Sep. 25, 2020

Related U.S. Application Data
(60) Provisional application No. 62/905,519, filed on Sep. 25, 2019.

(51) Int. Cl. G16B 25/10; G16B 40/00; G16B 5/00; G06F 7/58; G16H 10/40

(52) U.S. Cl. CPC G16B 25/10; G16B 40/00; G16H 10/40; G06F 7/588; G16B 5/00

(57) ABSTRACT

Method to process single cell gene expression data to reveal gene-gene correlations by applying a noise regularization process to reduce the gene-gene correlation artifacts. The computer-implemented method of the present application comprises processing gene expression data for normalization or imputation, applying a noise regularization process to the normalized or imputed gene expression data, and applying gene-gene correlation calculation process to obtain correlated gene pairs. Random noises based on an expression value of a gene in a cell in an expression matrix are added to obtain a noise regularized expression matrix.

### FIELD

The present invention generally pertains to methods and systems for processing gene expression data for gene-gene correlation by applying a noise regularization process.

### BACKGROUND

Gene expression data obtained from microarray and RNA sequencing of bulk cells has been successfully used to infer gene-gene correlations for constructing gene networks (Ballouz et al., Guidance for RNA-seq co-expression network construction and analysis: safety in numbers. Bioinformatics, 2015. 31(13): p. 2123-2130), but the analytic results of the expression data are limited to measuring average gene expression across pools of cells. The availability of single cell RNA sequencing (scRNA-seq) technology makes it possible to profile gene expression at the single cell resolution level, which then allows dissecting the heterogeneity within superficially homogenous cell populations to reveal hidden gene-gene correlations masked in bulk expression profiles (Kolodziejczyk et al., The Technology and Biology of Single-Cell RNA Sequencing. Molecular Cell, 2015. 58(4): p. 610-620; Papalexi et al., Single-cell RNA sequencing to explore immune cell heterogeneity. Nature Reviews Immunology, 2018. 18(1): p. 35).

However, there are challenges in processing scRNA-seq data due to technical limitations, such as dropout events and a high level of noise. Various approaches have been adopted to mitigate the noises caused by low efficiency and to estimate the true expression levels in processing scRNA-seq data. Numerous data preprocessing methods have been proposed as the first step of scRNA-seq data analysis. These data preprocessing methods may affect gene-gene correlation inference and subsequent gene co-expression network construction, such as introducing false positive gene-gene correlations.

It will be appreciated that a need exists for methods and systems for processing scRNA-seq data, which can efficiently reduce the gene-gene correlation artifacts for inferring gene-gene correlations and further constructing gene networks.

### SUMMARY

The availability of scRNA-seq data allows dissecting heterogeneity within homogenous cell populations to reveal hidden gene-gene interactions by profiling gene expression at the single cell resolution level. Challenges in processing scRNA-seq data can be due to technical limitations, such as dropouts (undetected gene expression) and high noises (variations). Data preprocessing methods have been adopted to mitigate the noise to estimate the true expression levels in processing scRNA-seq data. However, these data preprocessing methods may affect gene-gene correlation inference by introducing false positive gene-gene correlations.

The present application provides a method and system to process gene expression data for revealing gene-gene correlations by applying a noise regularization process to reduce gene-gene correlation artifacts. This disclosure also provides a method for improving data processing for gene-gene correlation, comprising: processing gene expression data for normalization or imputation, applying a noise regularization process to the normalized or imputed gene expression data, and applying a gene-gene correlation calculation process to obtain correlated gene pairs. In some exemplary embodiments, the gene expression data is single cell gene expression data. In some exemplary embodiments, the noise regularization process comprises adding a random noise to an expression value of a gene in a cell in an expression matrix and the random noise is determined by an expression level of the gene.

In some exemplary embodiments, the random noise is determined by: (1) determining an expression distribution of the gene across all of the cells in the expression matrix, (2) taking from about 0.1 to about 20 percentile of an expression level of the gene as a maximal noise level, (3) generating a random number ranging from 0 to the maximal noise level under uniform distribution, and (4) adding the random number to the expression value of the gene in the cell in the expression matrix to obtain a noise regularized expression matrix.

In some exemplary embodiments, the random noise is determined by: (1) determining an expression distribution of the gene across all of the cells in the expression matrix, (2) taking one percentile of an expression level of the gene as a maximal noise level, (3) generating a random number ranging from 0 to the maximal noise level under uniform distribution, and (4) adding the random number to the expression value of the gene in the cell in the expression matrix to obtain a noise regularized expression matrix.

In some exemplary embodiments, the gene-gene correlation calculation process is conducted with cell clusters. In some exemplary embodiments, Total Unique Molecular Identifier Normalization (NormUMI), Regularized Negative Binomial Regression (NBR), a deep count autoencoder network (DCA), Markov affinity-based graph imputation of cells (MAGIC), or single-cell analysis via expression recovery (SAVER) is used for processing gene expression data for normalization or imputation. In some exemplary embodiments, the method for improving data processing for gene-gene correlation of the present application further comprises enriching the gene expression data that is associated with the correlated gene pairs and/or constructing gene-gene correlation networks based on the correlated gene pairs, wherein the gene-gene correlation networks are cell type-specific. In some exemplary embodiments, the method of the present application further comprises using the gene-gene correlation networks for mapping molecular interactions, guiding experimental designs to investigate the biological events, discovering biomarkers, guiding comparative network analysis, guiding drug designs, identifying changes of gene-gene interactions by comparing healthy and disease states of cells, guiding drug development, predicting transcription regulation of genes, improving drug efficiency, or identifying drug resistance factors.

This disclosure, at least in part, provides a gene-gene correlation network, wherein the network is constructed based on correlated gene pairs which are obtained using the method for improving data processing for gene-gene correlation of the present application, and wherein the method comprises: processing gene expression data for normalization or imputation; applying a noise regularization process to the normalized or imputed gene expression data; and applying a gene-gene correlation calculation process to obtain correlated gene pairs.

This disclosure, at least in part, provides a computer-implemented method for data processing for gene-gene correlation, comprising: retrieving gene expression data; processing the gene expression data for normalization or imputation, applying a noise regularization process to the normalized or imputed gene expression data, applying a gene-gene correlation calculation process to obtain correlated gene pairs, and constructing gene-gene correlation networks based on the correlated gene pairs, wherein the gene-gene correlation networks are cell type-specific. In some exemplary embodiments, the gene expression data is single cell gene expression data. In some exemplary embodiments, the noise regularization process comprises adding a random noise to an expression value of a gene in a cell in an expression matrix and the random noise is determined by an expression level of the gene.

In some exemplary embodiments, the random noise is determined by: (1) determining an expression distribution of the gene across all of the cells in the expression matrix, (2) taking from about 0.1 to about 20 percentile of an expression level of the gene as a maximal noise level, (3) generating a random number ranging from 0 to the maximal noise level under uniform distribution, and (4) adding the random number to the expression value of the gene in the cell in the expression matrix to obtain a noise regularized expression matrix.

In some exemplary embodiments, the random noise is determined by: (1) determining an expression distribution of the gene across all of the cells in the expression matrix, (2) taking one percentile of an expression level of the gene as a maximal noise level, (3) generating a random number ranging from 0 to the maximal noise level under uniform distribution, and (4) adding the random number to the expression value of the gene in the cell in the expression matrix to obtain a noise regularized expression matrix.

In some exemplary embodiments, the gene-gene correlation calculation process is conducted with cell clusters. In some exemplary embodiments, Total Unique Molecular Identifier Normalization (NormUMI), Regularized Negative Binomial Regression (NBR), a deep count autoencoder network (DCA), Markov affinity-based graph imputation of cells (MAGIC), or single-cell analysis via expression recovery (SAVER) is used for processing gene expression data for normalization or imputation.

In some exemplary embodiments, the computer-implemented method for data processing for gene-gene correlation of the present application further comprises enriching the gene expression data that is associated with the correlated gene pairs. In some exemplary embodiments, the computer-implemented method of the present application further comprises using the gene-gene correlation networks for mapping molecular interactions, guiding experimental designs to investigate the biological events, discovering biomarkers, guiding comparative network analysis, guiding drug designs, identifying changes of gene-gene interactions by comparing healthy and disease states of cells, guiding drug development, predicting transcription regulation of genes, improving drug efficiency, or identifying drug resistance factors.

This disclosure, at least in part, provides a computer-based system for data processing for gene-gene correlation, comprising: a database configured to store gene expression data; a memory configured to store instructions; at least one processor coupled with the memory, wherein the at least one processor is configured to: retrieving the gene expression data, processing the gene expression data for normalization or imputation, applying a noise regularization process to the normalized or imputed gene expression data, applying a gene-gene correlation calculation process to obtain correlated gene pairs, and constructing gene-gene correlation networks based on the correlated gene pairs; and a user interface capable of receiving a query regarding data processing for gene-gene correlation and displaying the results of the correlated gene pairs and the constructed gene-gene correlation networks. In some exemplary embodiments, the gene expression data is single cell gene expression data and the gene-gene correlation networks are cell type-specific. In some exemplary embodiments, the noise regularization process comprises adding a random noise to an expression value of a gene in a cell in an expression matrix and the random noise is determined by an expression level of the gene.

In some exemplary embodiments, the random noise is determined by: (1) determining an expression distribution of the gene across all of the cells in the expression matrix, (2) taking from about 0.1 to about 20 percentile of an expression level of the gene as a maximal noise level, (3) generating a random number ranging from 0 to the maximal noise level under uniform distribution, and (4) adding the random number to the expression value of the gene in the cell in the expression matrix to obtain a noise regularized expression matrix.

In some exemplary embodiments, the random noise is determined by: (1) determining an expression distribution of the gene across all of the cells in the expression matrix, (2) taking one percentile of an expression level of the gene as a maximal noise level, (3) generating a random number ranging from 0 to the maximal noise level under uniform distribution, and (4) adding the random number to the expression value of the gene in the cell in the expression matrix to obtain a noise regularized expression matrix.

In some exemplary embodiments, the gene-gene correlation calculation process is conducted with cell clusters. In some exemplary embodiments, Total Unique Molecular Identifier Normalization (NormUMI), Regularized Negative Binomial Regression (NBR), a deep count autoencoder network (DCA), Markov affinity-based graph imputation of cells (MAGIC), or single-cell analysis via expression recovery (SAVER) is used for processing gene expression data for normalization or imputation. In some exemplary embodiments, the at least one processor is further configured to enrich the gene expression data that is associated with the correlated gene pairs.

In some exemplary embodiments, the at least one processor is further configured to utilize the gene-gene correlation networks for gene-gene correlation networks for mapping molecular interactions, guiding experimental designs to investigate the biological events, discovering biomarkers, guiding comparative network analysis, guiding drug designs, identifying changes of gene-gene interactions by comparing healthy and disease states of cells, guiding drug development, predicting transcription regulation of genes, improving drug efficiency, or identifying drug resistance factors.

These, and other, aspects of the invention will be better appreciated and understood when considered in conjunction with the following description and the accompanying drawings. The following description, while indicating various embodiments and numerous specific details thereof, is given by way of illustration and not of limitation. Many substitutions, modifications, additions, or rearrangements may be made within the scope of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a diagram for a computer-based system for data processing for improved gene-gene correlation, comprising a database, a memory, at least one processor and a user interface according to an exemplary embodiment.

FIG. 2 shows a flow chart for applying a noise regularization process to the normalized or imputed gene expression data according to an exemplary embodiment.

FIG. 3 shows a bone marrow scRNA-seq data from Human Cell Atlas Preview Datasets which was used as benchmarking dataset for various data preprocessing methods according to an exemplary embodiment. The full dataset contains 378,000 bone marrow cells which can be grouped into 21 cell clusters, covering all major immune cell types.

FIG. 4 shows an overview of a benchmarking framework according to an exemplary embodiment. Five representative data preprocessing methods, e.g., NormUMI, NBR, DCA, MAGIC, and SAVER, were applied to the single cell expression data matrix, e.g., bone marrow single cell expression data, according to an exemplary embodiment. Route 1 indicates the gene-gene correlations, which were calculated directly from the resulting matrix. Route 2 indicates the addition of a noise regularization step, wherein random noises determined by gene expression level (red areas) were applied to the expression matrix before proceeding to gene-gene correlation calculation. The enrichment of derived gene-gene correlations in protein-protein interaction (PPI) and the consistencies between methods were evaluated.

FIGS. 5A-5D show the observation of artifacts when five data preprocessing methods were used to process scRNA-seq data according to an exemplary embodiment. FIG. 5A shows that the distributions of correlation were different among these methods according to an exemplary embodiment. Lines indicates median.

FIG. 5B shows enrichment of top correlated gene pairs in protein-protein interaction for each method according to an exemplary embodiment. X-axis indicates the top n gene pairs. Y-axis indicates the fraction of the n gene pairs appearing in the STRING protein-protein interaction (PPI) database.

FIG. 5C shows that there were low consistencies among the methods in inferring the highly correlated gene pairs according to an exemplary embodiment.

FIG. 5D shows enrichment of randomly sampled gene pairs according to an exemplary embodiment.

FIG. 6 shows scatter plots of the expression values of the gene pair of MB21D1 and OGT, e.g., a negative gene control pair, after applying different data preprocessing methods according to an exemplary embodiment. Five representative data preprocessing methods, e.g., NormUMI, NBR, DCA, MAGIC, and SAVER, were applied in the analysis.

FIGS. 7A-7C show the results of applying noise regularization to reduce spurious correlation for five representative preprocessing methods, e.g., NormUMI, NBR, DCA, MAGIC, or SAVER, according to an exemplary embodiment. FIG. 7A shows the results of correlation distributions after applying noise regularization to each method according to an exemplary embodiment. Different colors indicate different methods.

FIG. 7B shows enrichment of top correlated gene pairs in protein-protein interaction after applying noise regularization according to an exemplary embodiment. X-axis indicates the top n gene pairs. Y-axis indicates the fraction of the n gene pairs appearing in the STRING protein-protein interaction (PPI) database. Different colors indicate different methods. Error bar in solid lines indicates 99% confidence interval based on 10 replicates.

FIG. 7C shows consistencies among the methods after applying noise regularization in inferring the highly correlated gene pairs according to an exemplary embodiment.

FIGS. 8A-8C show gene-gene correlation networks inferred from scRNA-seq data according to an exemplary embodiment. FIG. 8A and FIG. 8B show the comparison of Degree and Pagerank of each gene in the correlation networks constructed before and after applying noise regularization according to an exemplary embodiment.

FIG. 8C shows network construction with refined gene-gene correlations according to an exemplary embodiment. The scRNA-seq data were processed by applying NBR and noise regularization. The links which were not present in protein-protein interaction were removed.

FIG. 9 shows enrichment of top correlated gene pairs in Reactome pathways before and after applying noise regularization according to an exemplary embodiment. X-axis indicates the top n gene pairs. Y-axis indicates the fraction of the n gene pairs appearing in the same pathway in Reactome database. Dashed lines and solid lines represent before and after noise regularization, respectively.

FIG. 10 shows the results of determining the optimal noise level by testing maximal noises at different percentiles according to an exemplary embodiment.

FIG. 11 shows the generation of random noises ranging from about 0 to 1 percentile of gene expression level and the addition of random noises to the expression matrix according to an exemplary embodiment.

### DETAILED DESCRIPTION

Due to the availability of high-throughput gene expression data, it is possible to construct gene regulatory networks in large scale through statistical inference from gene expression data, e.g., assuming a statistical perspective by placing the data in the center of focus. Various statistical network inference methods, e.g., inference algorithms, have been used to estimate the interactions. Inferred gene regulatory networks provide information about regulatory interactions between regulators and their potential targets, such as gene-gene interactions, or potential protein-protein interactions in a complex. These inferred networks represent statistically significant predictions of molecular interactions obtained from large scale gene expression data. (Emmert-Streib et al., Gene regulatory networks and their applications: understanding biological and medical problems in terms of networks. Frontiers in Cell and Developmental Biology, 2014. 2(38)).

The inferred gene regulatory networks can be used to help solve biological and biomedical problems, such as serving as a causal map of molecular interactions, guiding experimental designs, discovering biomarkers, guiding comparative network analysis, or guiding drug designs (Emmert-Streib et al.). In addition, the constructed networks can be used to identify downstream interactions and provide guidance for conducting further downstream analysis, such as identifying changes of gene-gene interactions by comparing healthy and disease states of cells, which could potentially save time for drug development.

The inferred gene regulatory networks can be used to help solve biological and biomedical problems by serving as a causal map of molecular interactions, such as to derive novel biological hypothesis about molecular interactions or to predict the transcription regulation of genes. This information can be used to guide laboratory experiments to investigate biological events, since the predicted links are supposed to correspond to actual physical binding events between molecules. In addition, these inferred networks can be used to discover or study biomarkers for diagnostic, predictive, or prognostic purposes. For example, the network-based biomarkers can be used as statistical measures for diagnostic purposes for cancers, since cancer is a complex disorder relevant to various pathways rather than individual genes. Furthermore, when more inferred gene regulatory networks become available, it will be possible to guide comparative network analysis to understand changes of gene-gene interactions across different physiological or disease conditions. (Emmert-Streib et al.) Consequently, these inferred networks can guide a more efficient design of rational drugs, such as improving drug efficiency or identifying drug resistance factors.

A gene-gene co-expression network can be considered a gene regulatory network which is constructed from gene-gene correlations inferred from gene expression data, such as inferred from single cell RNA sequencing (scRNA-seq) data. The gene-gene co-expression networks can be constructed from different physiological, disease or treatment conditions. Comparing gene-gene co-expression networks constructed under different conditions will allow understanding gene interaction changes across different physiological or disease conditions to analyze such phenotypes under different conditions. For example, expression of two genes could be highly correlated in one cell type, but unrelated in other cell types. ScRNA-seq data can unbiasedly capture whole transcriptome of different cell types in a heterogenous cell population, which can reveal gene-gene correlation specific to certain cell types.

Gene expression is regulated by networks of transcription factors and signaling molecules. ScRNA-seq data can provide critical information for understanding cellular and tissue heterogeneity by revealing the dynamics of differentiation and quantifying gene transcription, since each cell is an independent identity representing different types or stages of biological events. Correlated expression, especially co-expression, between genes could be informative to build up networks for visualization and interpretation (Stuart et al., A Gene-Coexpression Network for Global Discovery of Conserved Genetic Modules. Science, 2003. 302(5643): p. 249-255). The analysis of scRNA-seq data can foster biological discoveries, because it can categorize each cell into different cell types or lineages to improve understanding of biological processes under different contexts. Therefore, gene-gene correlations revealed from single cell expression data have the potential to construct more comprehensive networks uncovering cell type specific modules.

Correlation metrics specifically tailored to single cell data were developed to analyze scRNA-seq data to infer large-scale regulatory networks under different organs and disease conditions. An unbiased quantification of a gene's biological relevance was computed using graph theory tools to pinpoint key players in organ function and drivers of diseases. (Iacono et al., Single-cell transcriptomics unveils gene regulatory network plasticity. Genome Biology, 2019. 20(1): p. 110). A genome-scale genetic interaction map was constructed by examining gene-gene pairs for synthetic genetic interactions. The network based on the genetic interaction profiles reveals a functional map by clustering similar biological processes in coherent subsets, wherein highly correlated profiles delineate specific pathways to define gene function (Costanzo, M., et al., The Genetic Landscape of a Cell. Science, 2010. 327(5964): p. 425-431).

However, there are challenges in utilizing scRNA-seq data due to technical limitations, such as dropout events (e.g., gene expression undetectable by scRNA-seq), a high level of noise (variations), and very large data volumes. In addition, only a small fraction of the transcripts present in each cell are sequenced in scRNA-seq, which leads to unreliable quantification of lowly—and moderately—expressed genes. A large proportion of genes, such as exceeding 90% of the gene populations, have zero or low read counts due to low capturing and sequencing efficiency. Although many of the observed zero counts reflect true zero expression, a considerable fraction of the counts can be due to technical limitations (Huang et al., SAVER: gene expression recovery for single-cell RNA sequencing. Nature Methods, 2018. 15(7): p. 539-542). In addition, the observed sequencing depth could vary dramatically among cells. Variations in cell lysis, reverse transcription efficiency, and molecular sampling during sequencing can also contribute to the variabilities (Hicks et al., Missing data and technical variability in single-cell RNA-sequencing experiments. Biostatistics, 2017. 19(4): p. 562-578).

Various data preprocessing methods have been adopted to mitigate the noises caused by low efficiency and to estimate the true expression levels in processing scRNA-seq data, including expression normalization and dropout imputation. Data normalization often is required to remove the technique noise while preserving the true biological signals. The high dropout rate of scRNA-seq refers to a large proportion of genes with zero count due to technical limitations in detecting the transcripts (Svensson et al., Power analysis of single-cell RNA-sequencing experiments. Nature Methods, 2017. 14: p. 381; Ziegenhain et al., Comparative Analysis of Single-Cell RNA Sequencing Methods. Molecular Cell, 2017. 65(4): p. 631-643.e4). In order to handle the dropouts to recover the true gene expression, various data imputation methods can be used to preprocess scRNA-seq data, such as cell clustering, detection of differentially expressed genes, and trajectory analysis (Tian et al., Benchmarking single cell RNA-sequencing analysis pipelines using mixture control experiments. Nature Methods, 2019. 16(6): p. 479-487).

There are challenges in applying imputation methods concerning false gene-gene correlation, since these methods are designed for reverse engineering gene networks to measure gene-gene correlations. Andrews et al. tested several imputation methods on a small simulation dataset and found that dropout imputation would generate false positive gene-gene correlations (Andrews, T. and M. Hemberg, False signals induced by single-cell imputation [version 1; peer review: 4 approved with reservations]. F1000Research, 2018, 7(1740)). Some representative scRNA-seq normalization/imputation methods for data preprocessing have influence on gene-gene correlation inferences by introducing spurious or inflated correlations due to data over-smoothing or over-fitting. These methods can introduce correlation artifacts for gene pairs which are not expected to be co-expressed. Since false signal and correlation artifacts might be introduced in the data processing, obtained gene pairs with highest correlations from these methods can have weak enrichments in protein-protein interactions.

In machine learning, adding noise to the data under certain conditions could increase robustness of the results by reducing overfitting (Bishop, Training with noise is equivalent to Tikhonov regularization. Neural computation, 1995. 7(1): p. 108-116; Neelakantan et al., Adding gradient noise improves learning for very deep networks. arXiv preprint arXiv:1511.06807, 2015; Smilkov et al., Smoothgrad: removing noise by adding noise. arXiv preprint arXiv:1706.03825, 2017).

This disclosure provides methods and systems to satisfy the aforementioned demands by providing methods and systems for processing scRNA-seq data utilizing a novel noise regularization method which can efficiently reduce the gene-gene correlation artifacts for inferring gene-gene correlations and further constructing gene networks. The gene-gene correlations derived after applying the noise regularization method of the present application can be used to construct a gene co-expression network. The resulting networks were validated at multiple levels to confirm the reliability of constructing the networks. The quality of inferred biological networks was assessed using known interactions in protein-protein interaction databases.

In some exemplary embodiments, a noise regularization method of the present application is implemented to process the preprocessed scRNA-seq data by adding uniformly distributed noise relative to each gene's expression level. The gene-gene correlations obtained by adding a noise regularization method of the present application can be used to reconstruct gene co-expression networks by reducing the artifacts in gene-gene correlations. In some exemplary embodiments, several known cell modules, such as immune cell modules, were successfully revealed, which were not visible in the absence of the noise regularization method of the present application. In some exemplary embodiments, when the noise regularization method of the present application was added, the cell type marker genes were rated higher in network topological properties, e.g., higher values of Degree and Pagerank, pinpointing their key roles in their respective cell clusters. The noise regularization method of the present application provides an advantage of increasing robustness of the data processing by reducing over-smoothing or over-fitting of expression data.

In some exemplary embodiments, the present application provides a computer-implemented method for improving data processing for gene-gene correlation, the method comprising: processing gene expression data for normalization or imputation; applying a noise regularization process to the normalized or imputed gene expression data; and applying gene-gene correlation calculation process to obtain correlated gene pairs. In some exemplary embodiments, the present application provides a computer-based system for data processing for gene-gene correlation, comprising: a database configured to store gene expression data; a memory configured to store instructions; at least one processor coupled with the memory, wherein the at least one processor is configured to: retrieve the gene expression data, process the gene expression data for normalization or imputation, apply a noise regularization process to the normalized or imputed gene expression data, apply a gene-gene correlation calculation process to obtain correlated gene pairs, and construct gene-gene correlation networks based on the correlated gene pairs; and a user interface capable of receiving a query regarding data processing for gene-gene correlation and displaying the results of the correlated gene pairs and the constructed gene-gene correlation networks.

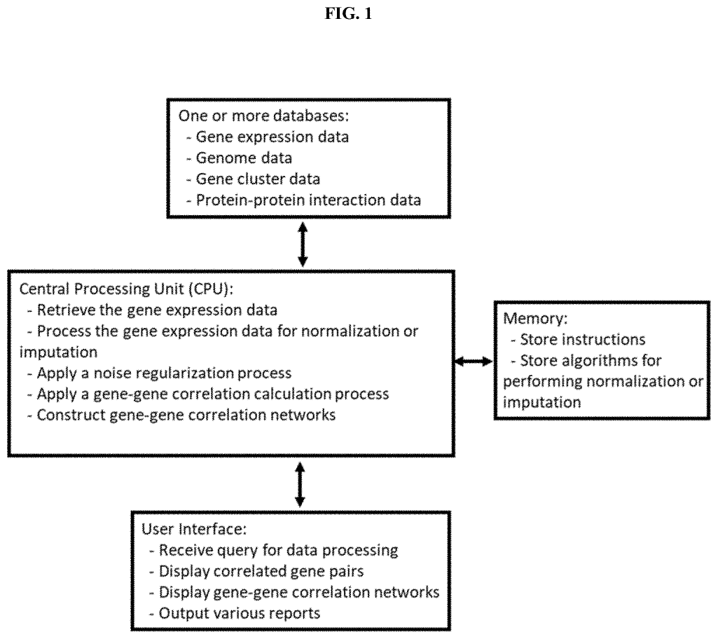

As shown in FIG. 1, an exemplary computer-based system of the present application for data processing for gene-gene correlation includes one or more databases, a central processing unit (CPU) comprising one or more processors, a memory coupled to CPU for storing instructions and a user interface. In some exemplary embodiments, the computer-based system of the present application further comprises algorithms for data normalization or imputation and various reports. In some exemplary embodiments, the databases include gene expression data, genome data or protein-protein interaction data. In some exemplary embodiments, the user interface can receive query for data processing, display correlated gene pairs or display gene-gene correlation networks.

In some exemplary embodiments, the random noise is determined by: (1) determining an expression distribution of the gene across all of the cells in the expression matrix, (2) taking one percentile of an expression level of the gene as a maximal noise level, (3) generating a random number ranging from 0 to the maximal noise level under uniform distribution, and (4) adding the random number to the expression value of the gene in the cell in the expression matrix to obtain a noise regularized expression matrix.

In some exemplary embodiments, the expression value of gene i in cell j is denoted as V, the random noise can be determined by: (i) calculating the expression distribution of gene i after applying various data preprocessing methods, (ii) determining the 1 percentile of expression value of gene i, which is denoted as M, wherein M will be used as the maximal of noise level, and (iii) generating a uniformly distributed random number, ranging from 0 to M, and adding this random number to V.

In some exemplary embodiments, random noise is generated and added to V, e.g., an expression value of gene i in cell j in the expression matrix which is processed by a specific method, wherein the random noise is determined by: (1) determining the expression distribution of gene i across all the cells, (2) taking one percentile of the gene i expression as the maximal noise level, denoted as M, (3) if M equals to zero, using 0.1 as the maximal noise level, (4) generating a random number ranging from 0 to M under uniform distribution, and (5) adding the random number to V to obtain the noise regularized expression matrix.

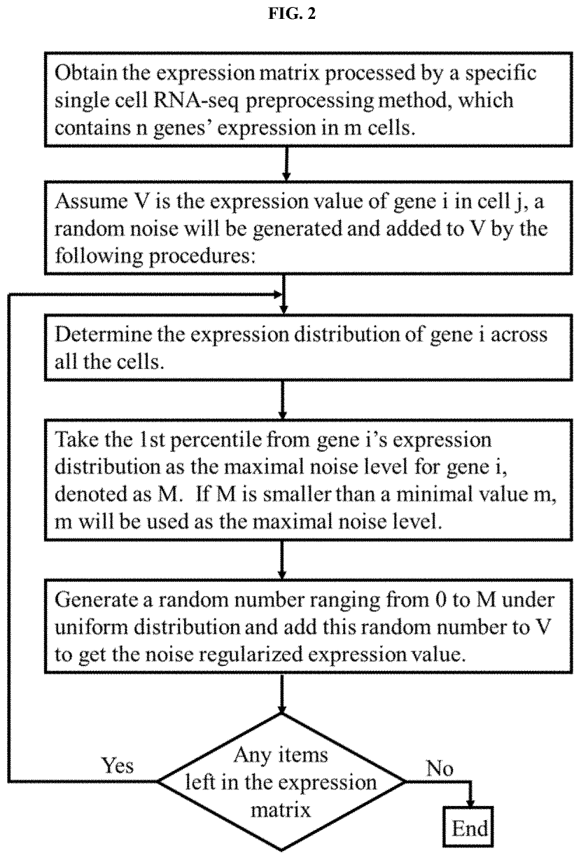

In some exemplary embodiments, the noise regularization process includes obtaining the expression matrix processed by a specific scRNA-seq preprocessing method, wherein this expression matrix contained n genes' expression in m cells. Assuming Vis the expression value of gene i in cell j, random noise is generated and added to V, wherein the random noise is determined by the following procedure: (1) determining the expression distribution of gene i across all the cells, (2) taking the 1st percentile from gene i's expression distribution as the maximal noise level for gene i, denoted as M, wherein if M is smaller than a minimal value m, m will be used as the maximal noise level, (3) generating a random number ranging from 0 to M under uniform distribution, (4) adding this random number to V to obtain the noise regularized expression value, and (5) repeating this procedure for every item in the expression matrix, as shown in the exemplary flow chart of FIG. 2.

Exemplary embodiments disclosed herein satisfy the aforementioned demands by providing computer-implemented methods to improve processing gene expression data for gene-gene correlation by applying a noise regularization process to the normalized or imputed gene expression data.

In some exemplary embodiments, computer-implemented methods are provided for improving data processing of gene expression data for gene-gene correlation by applying a noise regularization process to the normalized or imputed gene expression data. They satisfy the long felt needs of efficiently reducing the gene-gene correlation artifacts for inferring gene-gene correlations and further constructing gene networks.

The term “a” should be understood to mean “at least one”; and the terms “about” and “approximately” should be understood to permit standard variation as would be understood by those of ordinary skill in the art; and where ranges are provided, endpoints are included.

As used herein, the terms “include,” “includes,” and “including,” are meant to be non-limiting and are understood to mean “comprise,” “comprises,” and “comprising,” respectively.

In some exemplary embodiments, the disclosure provides a computer-implemented method for improving data processing for gene-gene correlation, comprising: processing gene expression data for normalization or imputation; applying a noise regularization process to the normalized or imputed gene expression data; and applying gene-gene correlation calculation process to obtain correlated gene pairs. In some exemplary embodiments, the noise regularization process is applied prior to applying the gene-gene correlation calculation process. In some exemplary embodiments, the gene expression data is single cell gene expression data.

As used herein, the term “gene-gene correlation” refers to pairs of genes which show a similar expression pattern across samples. When two genes are co-expressed, the expression levels of these two genes rise and fall together. Co-expressed genes are often involved in the same biological pathway, commonly regulated by the same transcription factor, or otherwise functionally related.

As used herein, the term “normalization” refers to a process of organizing a data set to reduce redundancy and improve data integrity including adding adjustments to bring the adjusted values into alignment or to fit certain distribution. Normalization process could remove systematic variations (e.g. variability in experiment conditions, machine parameters) and allow unbiased comparison across samples.

As used herein, the term “imputation” refers to a process of replacing missing data with substituted values. Missing data can cause problems of, for example, introducing a substantial amount of bias by creating reductions in efficiency which may affect the representativeness of the results. Imputation includes a process to substitute missing data with an estimated value based on other available information, which can enable the analysis of data sets using standard techniques.

### EXEMPLARY EMBODIMENTS

Embodiments disclosed herein provide methods to improve processing gene expression data for gene-gene correlation by applying a noise regularization process to normalized or imputed gene expression data.

In some exemplary embodiments, the disclosure provides a method for improving data processing to reduce gene-gene correlation artifacts, comprising: processing scRNA-seq data for normalization or imputation; applying a noise regularization process to the normalized or imputed gene expression data; and applying gene-gene correlation calculation process to obtain correlated gene pairs, wherein the noise regularization process comprises adding a random noise to an expression value of a gene in a cell in an expression matrix.

In some exemplary embodiments, the random noise is determined by: (1) determining an expression distribution of the gene across all of the cells in the expression matrix, (2) taking from about 0.1 to about 20 percentile of an expression level of the gene as a maximal noise level, (3) generating a random number ranging from 0 to the maximal noise level under uniform distribution, and (4) adding the random number to the expression value of the gene in the cell in the expression matrix to obtain a noise regularized expression matrix.

In some specific exemplary embodiments, the random noise is determined by: (1) determining an expression distribution of the gene across all of the cells in the expression matrix, (2) taking from about 0.1 to about 20 percentile, about 0.1 percentile, about 0.5 percentile, about 1 percentile, about 1.5 percentile, about 2 percentile, about 3 percentile, about 4 percentile, about 5 percentile, about 7 percentile, about 10 percentile, about 15 percentile, about 20 percentile, or about 25 percentile of an expression level of the gene as a maximal noise level, (3) generating a random number ranging from 0 to the maximal noise level under uniform distribution, and (4) adding the random number to the expression value of the gene in the cell in the expression matrix to obtain a noise regularized expression matrix, wherein the computer-implemented method of the present application further comprises constructing gene-gene correlation networks based on the correlated gene pairs.

In some exemplary embodiments, the computer-implemented method of the present application further comprises using the gene-gene correlation networks for mapping molecular interactions, guiding experimental designs to investigate the biological events, discovering biomarkers, guiding comparative network analysis, guiding drug designs, identifying changes of gene-gene interactions by comparing healthy and disease states of cells, guiding drug development, predicting transcription regulation of genes, improving drug efficiency, identifying drug resistance factors, providing guidance for conducting further downstream analysis, deriving novel biological hypothesis about molecular interactions, providing statistical measures for diagnostic purposes for cancers, guiding comparative network analysis to understand changes of gene-gene interactions across different physiological or disease conditions, understanding gene interaction changes to analyze specific phenotypes under different conditions, revealing dynamics of differentiation for quantifying gene transcription, or discovering biomarkers for diagnostic, predictive, or prognostic purposes.

It is understood that the method or system is not limited to any of the aforesaid methods or systems to improve processing gene expression data for gene-gene correlation. The consecutive labeling of method steps as provided herein with numbers and/or letters is not meant to limit the method or any embodiments thereof to the particular indicated order. Various publications, including patents, patent applications, published patent applications, accession numbers, technical articles and scholarly articles are cited throughout the specification. Each of these cited references is incorporated by reference, in its entirety and for all purposes, herein. Unless described otherwise, all technical and scientific terms used herein have the same meaning as commonly understood by one of ordinary skill in the art to which this invention belongs.

The disclosure will be more fully understood by reference to the following Examples, which are provided to describe the disclosure in greater detail. They are intended to illustrate and should not be construed as limiting the scope of the disclosure.

### EXAMPLES

### Databases and Methods

Obtain scRNA-Seq Datasets

Bone marrow scRNA-seq data was retrieved from Human Cell Atlas Data Portal (https://preview.data.humancellatlas.org/). The retrieved datasets contain profiling data for 378,000 immunocytes by 10× platform. In order to reduce the computational burden, 50,000 cells were randomly sampled from the original datasets. Subsequently, genes expressed in less than 100 cells (0.2%) were further filtered out. In the output, 12,600 genes remained in the final benchmarking datasets. Single cell analysis, such as clustering or dimension reduction, was performed using Seurat R package Version 3.0.

Data Normalization or Imputation

Several methods were applied in a data pre-processing step for data normalization or imputation, including Total Unique Molecular Identifier Normalization (NormUMI), Regularized Negative Binomial Regression (NBR; Hafemeister et al., Normalization and variance stabilization of single-cell RNA-seq data using regularized negative binomial regression. bioRxiv, 2019: p. 576827), a deep count autoencoder (DCA) network (Eraslan et al., Single-cell RNA-seq denoising using a deep count autoencoder. Nature Communications, 2019. 10(1): p. 390), Markov affinity-based graph imputation of cells (MAGIC; van Dijk, et al., Recovering Gene Interactions from Single-Cell Data Using Data Diffusion. Cell, 2018. 174(3): p. 716-729.e27), or single-cell analysis via expression recovery (SAVER; Huang et al.). NBR, SAVER and DCA were run with default parameters following the tool instructions. MAGIC was run with following parameters: number of principle component npca=30, the power of the Markov affinity matrix t=6 and number of nearest neighbor k=30. NormUMI and NBR are normalization methods. DCA, MAGIC and SAVER methods are imputation methods.

Gene-Gene Correlation Calculation

Spearman correlations of each gene pair were calculated within cells in each cluster, such as from cluster 0 to cluster 9 respectively. A gene will be considered as expressed in one cluster, if it is expressed in greater than 1% cells or 50 cells in that cluster, whichever is greater. The correlation of a gene pair in one cluster was considered as an effective correlation, when both genes were expressed in the cluster. The highest effective correlation across the ten clusters (clusters 0-9) were recorded as the final correlation for a given gene pair.

Data Enrichment According to Protein-Protein Interaction

Human protein-protein interaction (PPI) data was retrieved from STRING database (http://string-db.org) (Szklarczyk, et al., STRING v10: protein—protein interaction networks, integrated over the tree of life. Nucleic Acids Research, 2014. 43(D1): p. D447-D452). Gene pairs were ranked by Spearman correlation coefficients for each method. Gene pairs with high ranks (top n gene pairs) were then taken and counted the fraction of the pairs appearing in protein-protein interaction database.

Noise Regularization

Noise regularization was applied for data processing. Random noises determined by gene expression level are added to the expression matrix before proceeding to correlation calculation. Random noise is generated and added to V, e.g., an expression value of gene i in cell j in the expression matrix which is processed by a specific method. Random noise is generated by (1) determining the expression distribution of gene i across all the cells, (2) taking one percentile of the gene i expression as the maximal noise level, denoted as M, (3) if M equals to zero, using 0.1 as the maximal noise level, (4) generating a random number ranging from 0 to M under uniform distribution, and (5) adding the random number to V to obtain the noise regularized expression matrix.

Network Construction

Spearman correlations of each gene pair were calculated within cells in each cluster. Within each cluster, the gene pairs were ranked by their Spearman correlations. Since housekeeping genes are required for basic cellular functions, they are expected to be expressed in all cells irrespective of tissue type or cell types. In order to construct cell type-specific interaction modules, housekeeping genes were removed from the network construction. The list of housekeeping genes which were removed included a housekeeping gene list which was obtained from Eisenberg et al. (Eisenberg et al., Human housekeeping genes, revisited. Trends in Genetics, 2013. 29(10): p. 569-574). In addition, typical housekeeping genes, such as ACTB, B2M, and ribosomal, TCA, cytoskeleton genes from Reactome, and mtDNA encode genes were added to the list of the housekeeping genes which were removed. After removing housekeeping genes, the gene pairs ranked in the top 1,000 from each cluster were taken and put together to construct the draft network. The importance of each node in the network was measured by the values of Degree and Pagerank using igraph R package according to Csardi et al. (Csardi et al., The igraph software package for complex network research. InterJournal, Complex Systems, 2006. 1695(5): p. 1-9). Subsequently, the network was cleaned by removing the links which were not referring to a protein-protein interaction in STRING database. The final network was visualized using Cytoscape according to Shannon et al. (Shannon et al., Cytoscape: A Software Environment for Integrated Models of Biomolecular Interaction Networks. Genome Research, 2003. 13(11): p. 2498-2504) together with R package RCy3 according to Ono et al. (Ono et al., CyREST: Turbocharging Cytoscape Access for External Tools via a RESTful API. F1000Research, 2015. 4: p. 478-478). The network layout was generated using EntOptLayout Cytoscape plug-in according to Agg et al. (Agg et al., The EntOptLayout Cytoscape plug-in for the efficient visualization of major protein complexes in protein—protein interaction and signaling networks. Bioinformatics, 2019).

### Example 1. Data Preprocessing Using Representative Normalization/Imputation Methods

Several representative normalization/imputation methods were benchmarked with a focus on their influences on gene-gene correlation inferences. Global scaling normalization methods had the least data manipulation through normalizing the gene expression for each cell by the total expression. This method is usually followed by log transformation and z-score scaling, since log transformation and z-score scaling will not change rank-based correlation; only Total UMI normalization was included in the comparison (referred to as NormUMI). A framework utilizing “Regularized Negative Binomial Regression” to normalize and stabilize variance of scRNA-seq data (referred as NBR) was included, which can remove the influence of technical noise while preserving biological heterogeneity. Three additional methods representing different imputation methodology categories were also included, e.g., (i) MAGIC—is a data smoothing approach which leverages the shared information across similar cells to de-noise and fill in dropout values; (ii) SAVER—a model based approach which models the expression of each gene under a negative binomial distribution assumption and outputs the posterior distribution of the true expression; and (iii) DCA—a deep learning based autoencoder to capture the complexity and non-linearity in scRNA-seq data and reconstruct the gene expressions.

These five exemplary normalization/imputation methods, e.g., NormUMI, NBR, DCA, MAGIC, and SAVER, were applied on bone marrow scRNA-seq data from Human Cell Atlas Project (Regev et al., The Human Cell Atlas. eLife, 2017. 6: p. e27041) by comparing the gene-gene correlations derived from the preprocessing methods. Except for NormUMI, the other four methods presented noticeable inflations of gene-gene correlations by introducing correlation artifacts for gene pairs which are not expected to be co-expressed. The gene pairs with highest correlations from these methods had weak enrichments in protein-protein interactions, suggesting that there might be false signal and correlation artifacts introduced in the data preprocessing. The false signals could be introduced by data preprocessing due to over-smoothing or over-fitting.

### Example 2. Calculate Gene-Gene Correlations in Single Cell

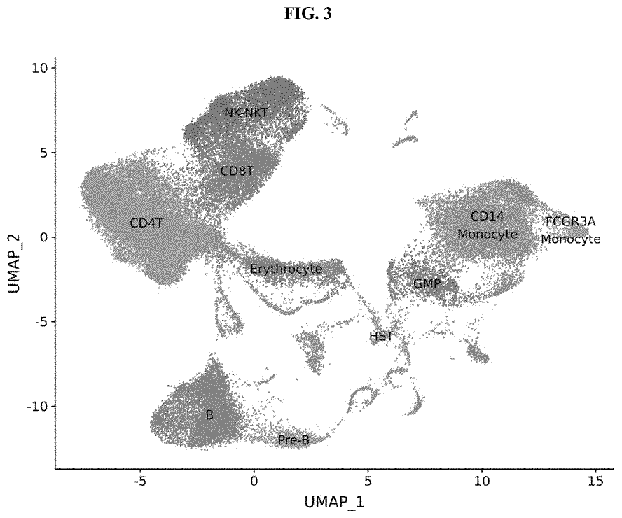

Real bone marrow scRNA-seq data from Human Cell Atlas Preview Datasets was used as benchmarking dataset (Regev et al.) for various data preprocessing methods. The full dataset contained 378,000 bone marrow cells which can be grouped into 21 cell clusters as shown in FIG. 3 and Table 1, covering all major immune cell types. 50,000 cells from the original dataset were randomly sampled. Genes expressing in less than 0.2% (100 cells) were excluded in this subset. The final dataset contained 12,600 genes, and resulted in over 79 million possible gene pairs.

| Cluster | 0 | 1 | 2 | 3 | 4 | 5 | 6 | 7 | 8 | 9 |
|---|---|---|---|---|---|---|---|---|---|---|
| Cell type | CD4T | CD14 | B | NK-NKT | CD8T | Erythrocyte | GMP | Pre-B | FCGR3A | HST |
| | | monocyte | | | | | | | monocyte | |
| Cell | 16936 | 7413 | 6534 | 5847 | 4467 | 1974 | 1347 | 1052 | 583 | 598 |
| number | | | | | | | | | | |
| Top 10 | IL7R | S100A9 | CD79A | GNLY | GZMK | HBB | MPO | CD79B | LST1 | SPINK2 |
| markers | LTB | S100A8 | CD74 | NKG7 | RGS1 | AHSP | ELANE | HIST1H1C | IFITM3 | AVP |
| | TRAC | S100A12 | IGHD | GZMB | CCL4 | CA1 | PRTN3 | TCL1A | AIF1 | SOX4 |
| | NOSIP | LYZ | MS4A1 | FGFBP2 | DUSP2 | HBD | AZU1 | SOX4 | FCGR3A | KIAA0125 |
| | LEPROTL1 | FCN1 | IGHM | GZMH | CMC1 | PRDX2 | LYZ | VPREB3 | COTL1 | ANKRD28 |
| | PIK3IP1 | CXCL8 | HLA- | PRF1 | CCL5 | HBA1 | CTSG | CD24 | FCER1G | IGLL1 |
| | | | DQB1 | | | | | | | |
| | CD3D | TYROBP | HLA- | CST7 | GZMA | BLVRB | RETN | NEIL1 | SERPINA1 | PRSS57 |
| | | | DRA | | | | | | | |
| | LDHB | VCAN | HLA- | KLRD1 | CST7 | HBA2 | RNASE2 | IGHM | S100A11 | PRDX1 |
| | | | DRB1 | | | | | | | |
| | MAL | CSTA | HLA- | CCL5 | IL32 | TUBA1B | LGALS1 | PCDH9 | SAT1 | H2AFY |
| | | | DPA1 | | | | | | | |
| | CD3E | NAMPT | HLA- | KLRF1 | KLRB1 | TUBB | H2AFZ | VPREB1 | PSAP | SERPINB1 |
| | | | DQA1 | | | | | | | |

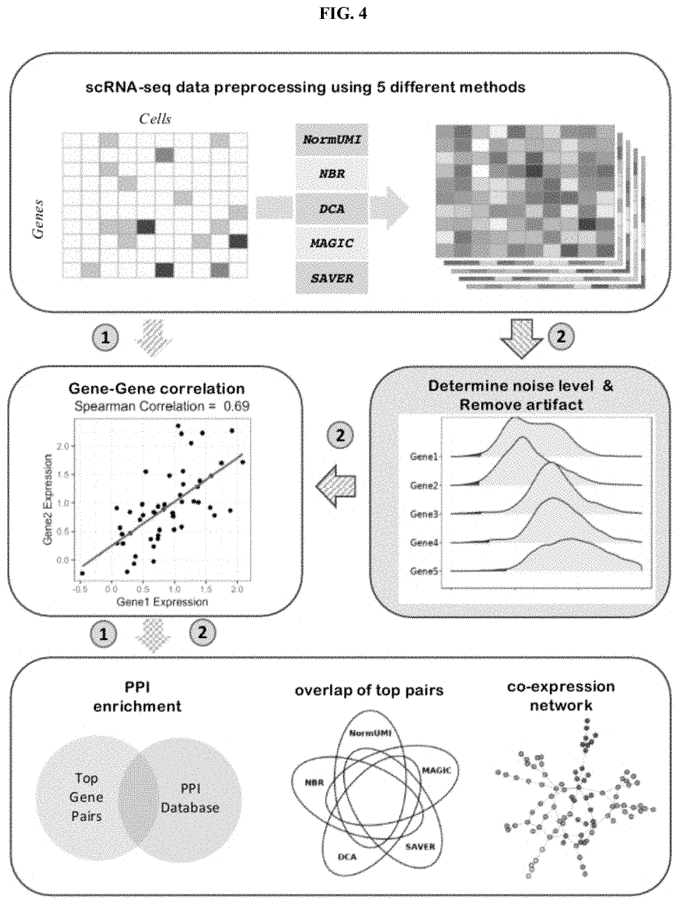

FIG. 4 shows an overview of the benchmarking framework. Five representative data preprocessing methods, e.g., NormUMI, NBR, DCA, MAGIC, and SAVER, were applied to the single cell expression data matrix, e.g., bone marrow single cell expression data, as shown in FIG. 4. The gene-gene correlations were calculated directly from the resulting matrix (denoted as route 1). The enrichment of derived gene-gene correlations in protein-protein interaction and the consistency between methods were evaluated. It was discovered that the data preprocessing procedure can introduce artificial correlations. A noise regularization step (denoted as route 2) was introduced, wherein random noises determined by gene expression level (red areas) were applied to the expression matrix before proceeding to correlation calculation. This noise regularization step effectively reduced the spurious correlations, and the refined gene-gene correlation metrics could be used to construct gene co-expression networks.

Expression of two genes could be highly correlated in one cell type, but unrelated in other cell types. To capture the gene-gene correlations across different cell types, the gene-gene spearman correlations were calculated within ten biggest clusters, e.g., greater than 500 cells per cluster, in benchmarking dataset, which includes CD4 T cell, CD8 T cell, natural killer cell, B cell, pre-B cell, CD14+ monocytes, FCGR3A+ monocytes, erythrocyte, granulocyte-macrophage progenitors and hematopoietic stem cells (FIG. 3 and FIG. 4). For each pair of genes, the highest correlation among the 10 clusters was recorded as the final correlation.

### Example 3. Observation of Artifacts Using Data Preprocessing Methods

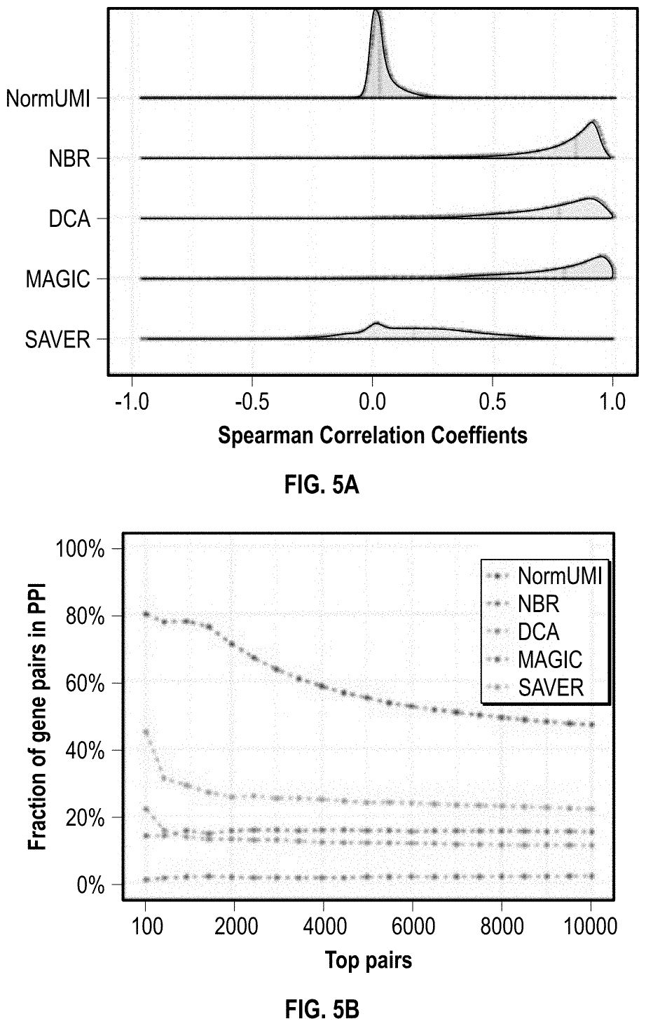

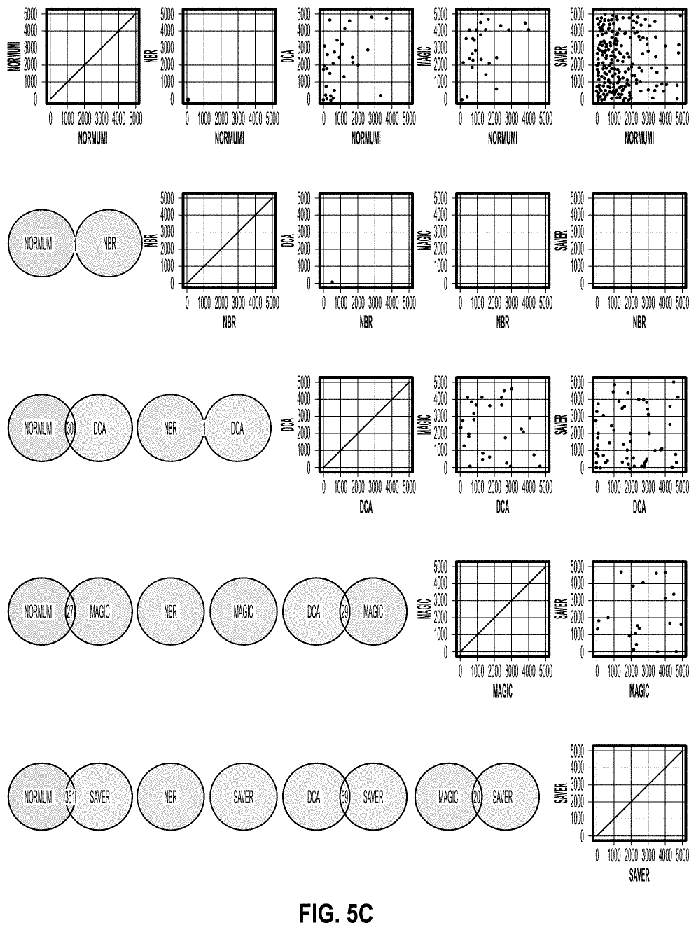

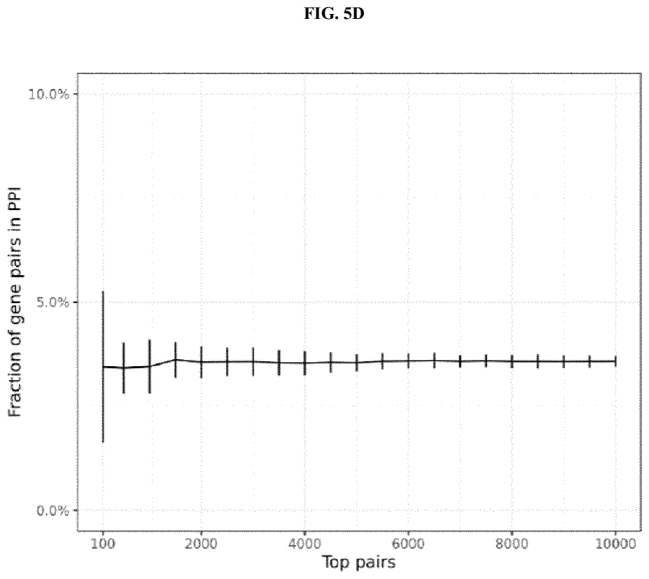

Five representative data preprocessing methods, e.g., NormUMI, NBR, DCA, MAGIC, and SAVER, were applied on bone marrow scRNA-seq data from Human Cell Atlas Project. The distributions of the overall gene-gene correlations in five different data matrices processed by different methods were compared. Since most of the gene pairs were not expected to have any association, the correlation distribution was anticipated to peak at 0. NormUMI produced a correlation distribution peaked at 0 as shown in FIG. 5A. However, the other four methods produced a much higher median correlation in terms of Spearman correlation coefficients as shown in FIG. 5A (NormUMI p=0.023, NBR p=0.839, MAGIC p=0.789, DCA p=0.770, SAVER p=0.166).

The interactions between two genes were accessed to reveal whether higher correlation would reflect a higher chance of either functional or physical interaction between two genes after applying a specific data preprocessing method. Proteins encoded by co-expressed genes are more frequently interacting with each other than a random protein pair. If the resulting higher correlations are true, the co-expressed genes should have relative higher enrichment in protein-protein interactions database, while spurious correlations should dilute the enrichment. STRING database (Szklarczyk et al.) which contains 5,772,157 interacting gene pairs was used to evaluate the protein-protein interaction enrichment in the top-ranked co-expressed gene pairs. Top gene pairs (by correlation ranking) from each method were selected. The fraction of these pairs that overlap with STRING database were calculated as shown in FIG. 5B. The results indicated that NormUMI had the highest protein-protein interaction enrichment at 80% and 47% overlap with STRING in the top 100 and 10,000 gene pairs, respectively. In contrast, the top gene pairs from NBR had lower than the expected overlap with STRING (<2%), while MAGIC and DCA had similar protein-protein interaction enrichment ranging from 11% to 22%. SAVER showed relative better results, but the enrichment was merely half of those of NormUMI.

Gene pairs were randomly sampled and overlapped the random pairs with PPI to estimate the background enrichment level (FIG. 5D). The estimated background enrichment level was about 3.6%, indicating that PPI enrichment of NBR was even lower than the background. Although this straightforward method directly relates physical interactions with gene coexpression, the results also provide a useful comparison among the data preprocessing methods given that the same assumption is made for all of them.

FIGS. 5A-5C show the results of observing artifacts, such as spurious gene-gene correlations, when data preprocessing methods were used to process gene expression data. The distributions of correlations were different among these methods as shown in FIG. 5A. NormUMI had a distribution centered close to zero, while NBR, DCA and MAGIC had apparent inflated correlation distributions. Lines indicates median. FIG. 5B shows enrichment of top correlated gene pairs in protein-protein interaction for each method. X-axis indicates the top n gene pairs. Y-axis indicates the fraction of the n gene pairs appearing in the STRING protein-protein interaction database. NormUMI had the highest enrichment, followed by SAVER, MAGIC, DCA and NBR. FIG. 5C shows that there were low consistencies among the methods in inferring the highly correlated gene pairs. Lower triangle indicates the overlapping of the top 5000 gene pairs between the methods. This highest overlapping was between NormUMI and DCA. Only 30 gene pairs ranked top 5,000 in both methods. Upper triangle compared the exact rank of the shared pairs between methods, showing low agreements.

The consistency of highly correlated gene pairs derived from the five data preprocessing procedures was compared. Pairwise comparison of the top 5,000 gene pairs from each method was performed. The results indicated that the overlapping of gene pairs between methods was minimal. For example, only one gene pair was shared by NormUMI and NBR out of the top 5,000 pairs. The highest overlapping was between NormUMI and DCA, which showed only 30 gene pairs shared by the two methods (lower triangle in FIG. 5C). The ranks of the overlapping pairs in each method were further compared. The results indicated that there was no well-defined or clear relationship according to these methods (upper triangle in FIG. 5C). Even though this approach did not provide a fully quantitative result, it indicated that the high correlations derived from these data preprocessing methods were likely to be artifacts.

### Example 4. Unrelated Genes as Negative Control Gene Pairs

Negative control gene pairs were used to investigate the potential causes of the spurious correlations. Negative control gene pairs were defined by the following criteria: (i) the two genes should not appear as an interacting pair in STRING database; (ii) the two genes should not share any gene ontology (GO) term (Ashburner et al., Gene ontology: tool for the unification of biology. The Gene Ontology Consortium. Nature genetics, 2000. 25(1): p. 25-29; The Gene Ontology Consortium, The Gene Ontology Resource: 20 years and still going strong. Nucleic Acids Research, 2018. 47(D1): p. D330-D338); and (iii) the two genes should not be on the same chromosome.

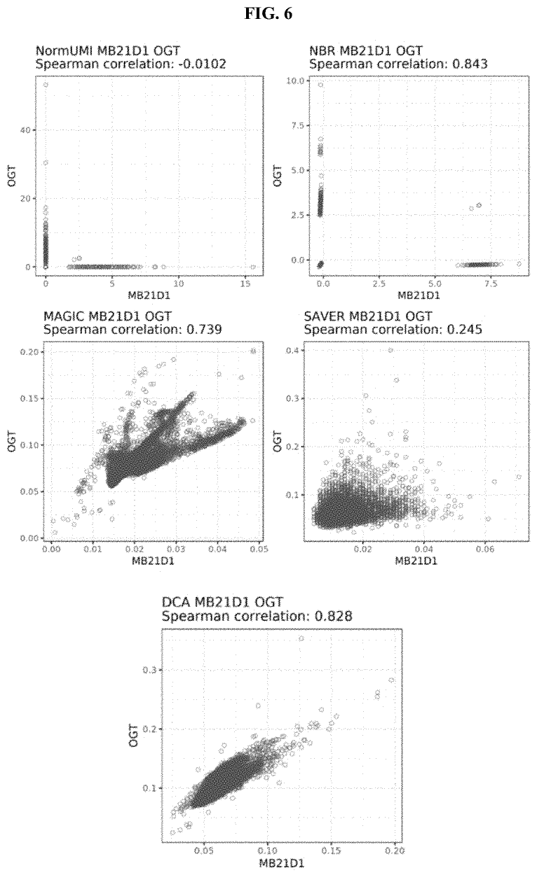

Scatter plots of the expression values of the gene pair of MB21D1 and OGT, e.g., a negative gene control pair, after applying different data preprocessing methods are shown in FIG. 6. There was no existing evidence indicating the correlation of these two genes. Only three out of 6534 cells in cluster 2 had non-zero expression value in both genes in the original expression matrix. Five representative data preprocessing methods, e.g., NormUMI, NBR, DCA, MAGIC, and SAVER, were applied to the analysis. One of the negative control gene pairs, MB21D1 and OGT, had high correlation after applying NBR (p=0.843), DCA (p=0.828), or MAGIC (p=0.739) processing method in cell cluster #2. The visualization suggested these correlation artifacts may be caused by data over-smoothing.

Out of the five methods, NormUMI was the only method that remains zero counts from the raw data. In the analysis using NormUMI, 6,110 cells out of 6,534 cells (93.5%) had zero values in both genes, 3 cells (0.04%) had non-zero values in both genes, while 1.3% and 5.2% cells had non-zero for MB21D1 and OGT, respectively. The other four methods intensely altered the zeros from the original expression matrix. After applying these procedures, all of the processed data presented some degree of over-smoothing, especially in the “double zeros regions” in the original data, which created the correlation artifact as shown in FIG. 6. Although NBR is not an imputation method and only shifted the zero values minimally, artificial rank correlation was introduced due to the different adjusted magnitude per cell.

### Example 5. Applying Noise Regularization Method to Reduce Spurious Correlation

A noise regularization method was applied to reduce spurious correlation. Random noises were added to every single item in the expression matrix processed by the preprocessing method, e.g., NormUMI, NBR, DCA, MAGIC, and SAVER. As an example, the expression value of gene i in cell j is denoted as V. The noises were generated by the following steps: (i) calculate the expression distribution of gene i after various data preprocessing methods; (ii) determine the 1 percentile of expression value of gene i, which is denote as M, M will be used as the maximal of noise level; and (iii) generate a uniformly distributed random number, ranging from 0 to M, and add this random number to V.

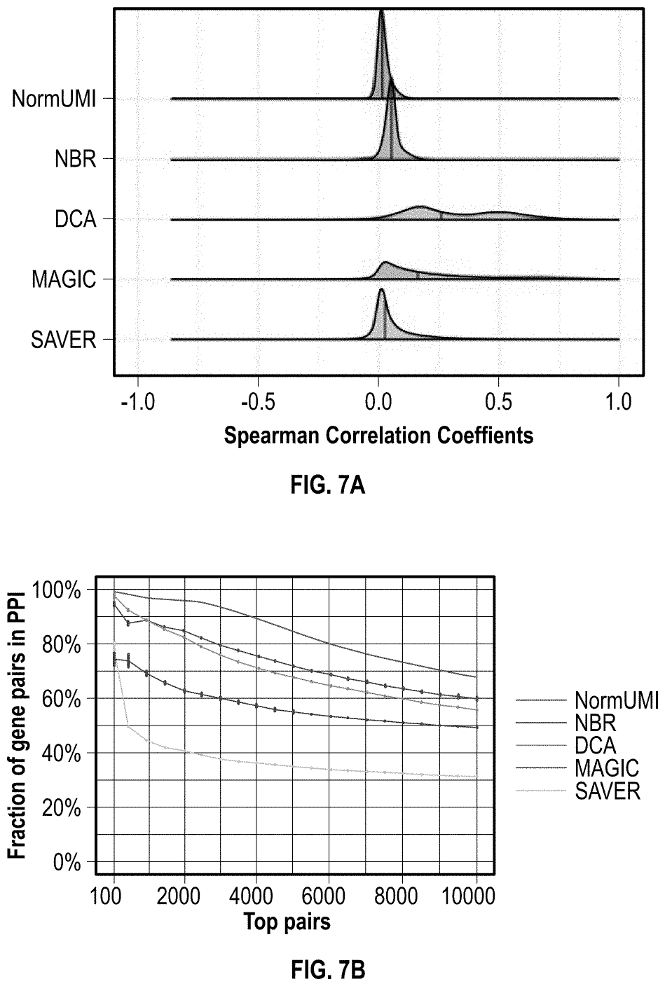

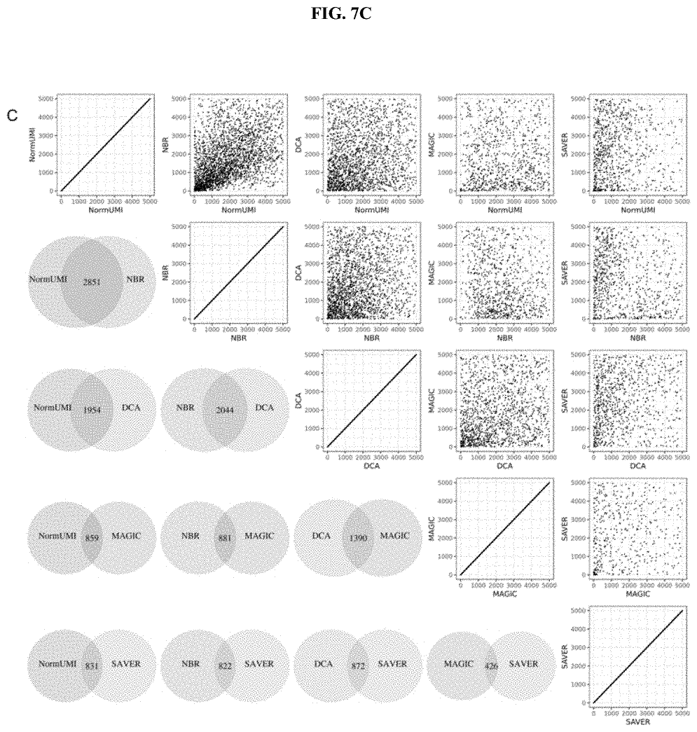

After applying this noise regularization method to each preprocessing method, the gene-gene correlations were recomputed. FIG. 7A shows the results of Spearman correlation analysis, e.g., correlation distributions, after applying noise regularization to each method according to an exemplary embodiment. Different colors indicate different methods. The results show that the correlation median shift towards 0 in all five methods as shown in FIG. 7A regarding distributions of correlation, which indicates a reduction in the correlation inflation due to the application of noise regularization.

FIG. 7B shows enrichment of top correlated gene pairs in protein-protein interaction after applying noise regularization according to an exemplary embodiment. X-axis indicates the top n gene pairs. The Y-axis indicates the fraction of the n gene pairs appearing in the STRING protein-protein interaction database. Different colors indicate different methods. The error bar in solid lines indicates 99% confidence interval based on 10 replicates. There were substantial improvements of the protein-protein interaction enrichment in the top correlated genes in all methods. NBR previously had the lowest enrichment in protein-protein interaction. However, after applying the noise regularization method, NBR shows the highest enrichment in protein-protein interaction. In the top 100, 1,000 and 10,000 correlated gene pairs in NBR, 99.0%, 96.8% and 67.7% of the gene pairs can be found in protein-protein interaction database, corresponding to 99.0-, 50.9- and 31.6-fold improvement, respectively. DCA on average had about 12% protein-protein interaction enrichment in previous results. After noise regularization, DCA had about 97.6% enrichment in the top 100 pairs and about 55.8% in the top 10,000 pairs, corresponding to about a 5-fold improvement. NormUMI which showed highest enrichment previously, also had about 1.1 to 1.3-fold improvements. To test whether these results of noise regularization are robust and reproducible, the procedures were repeated ten times with different random seeds to generate the random noises. The protein-protein interaction enrichment performances were stable between each repeat. The standard deviation of NBR in most points were less than 0.1% (error bar represents 99% confidence interval in FIG. 7B).

FIG. 7C shows consistencies among the methods after applying noise regularization in inferring the highly correlated gene pairs. There were more overlapping gene pairs between different methods. Among the top 5,000 gene pairs, there were 2,851 (57%) overlapped pairs between NormUMI and NBR (FIG. 7C lower triangle) and there was a significant correlation between the overlapped gene pairs (Spearman correlation=0.50, P value=1.77e−181, FIG. 7C upper triangle). Among other methods, it also showed some agreement, especially between the highly ranked genes. Comparing to the results which were generated without applying noise regularization as shown in FIG. 5C, there were higher agreements among different methods as shown in FIG. 7C. For example, more than 50% of gene pairs were shared between NormUMI and NBR after applying the noise regularization.

### Example 6. Gene-Gene Correlation Network Inferred from scRNA-Seq Data

Gene-gene correlations revealed from scRNA-seq can be used to reconstruct more comprehensive networks uncovering cell type specific modules. The combination of NBR and noise regularization of the present application as described in previous examples generated the highest protein-protein interaction enrichment among all the methods. Therefore, the gene-gene correlations which were derived by applying NBR and noise regularization of the present application to the scRNA-seq data as described in previous examples were used to reconstruct the gene-gene correlation network.

Since house-keeping genes typically reflect the basic and general cellular functions, in order to focus more on cell type specific interactions, house-keeping genes involving links were removed from the network construction. The top 1,000 gene pairs with highest correlations were taken from each cluster (cluster #0 to cluster #9) to reconstruct the network. Degree, Pagerank, the two algorithms from graph theory were used to measure the importance of each gene in the network. The value of Degree of a gene in the network equals to the number of links (interactions) that the gene has (Bondy et al., Graph Theory. 2008: Springer Publishing Company, Incorporated. 654). Important genes tend to connect with more genes, therefore important genes should have relative higher value of Degrees. In addition to the quantity of links, Pagerank is considered as evaluating the quality of links to a gene by measuring the overall popularity of a gene (Page et al., The PageRank citation ranking: Bringing order to the web. 1999, Stanford InfoLab).

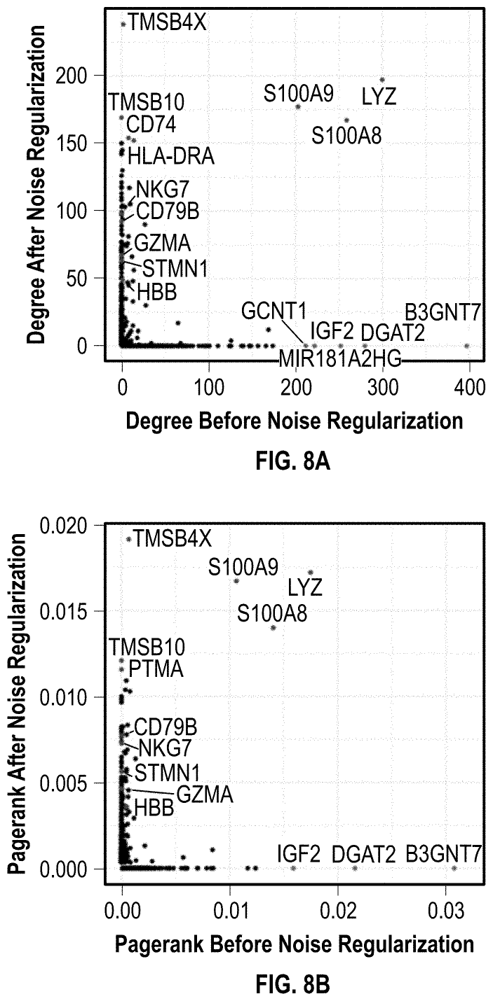

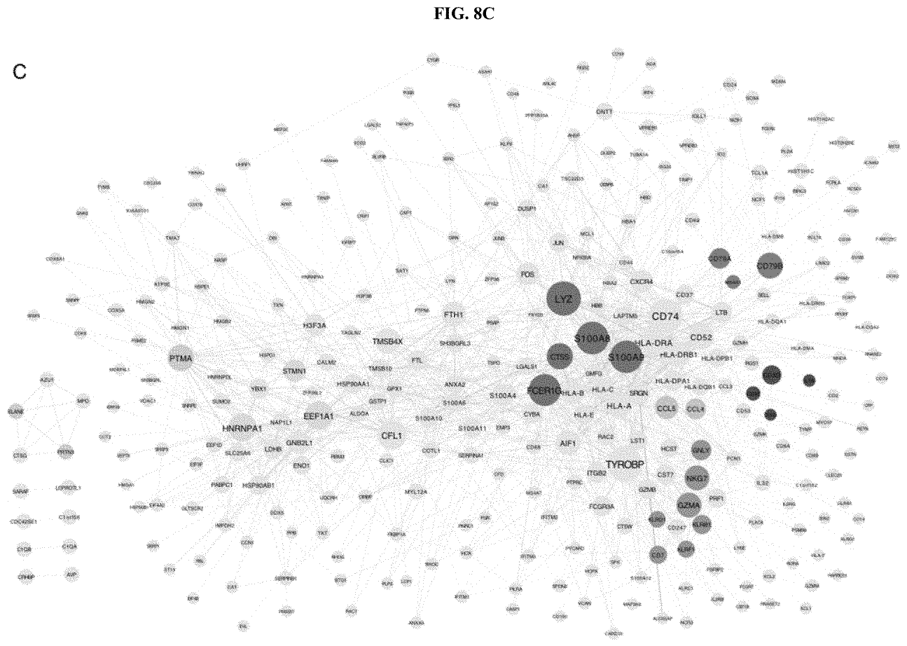

Comparing to the network constructed without noise regularization, networks constructed with the addition of noise regularization can better present the biological functions in topological structure. Furthermore, genes with higher values of Degree or Pagerank also tend to have important functions in the immune system. For example, LYZ, CD79B and NKG7 are important marker genes for monocytes, B cells and natural killer cells, respectively. These three genes had high values of Pagerank and Degree in the network with noise regularization. In contrast, CD79B and NKG7 did not exist in the network at all, if noise regularization was not applied as shown in FIG. 8A and FIG. 8B. Furthermore, known protein-protein interaction information was used to further refine the network (Cheng et al., Inferring Transcriptional Interactions by the Optimal Integration of ChIP-chip and Knock-out Data. Bioinformatics and biology insights, 2009. 3: p. 129-140; Sayyed-Ahmad et al., Transcriptional regulatory network refinement and quantification through kinetic modeling, gene expression microarray data and information theory. BMC Bioinformatics, 2007. 8(1): p. 20). Only gene-gene correlations which can be found in the STRING protein-protein interaction database were retained. Subsequently, EntOptLayout (Agg et al.) was applied. EntOptLayout is a network algorithm which provides an efficient visualization of different modules in the network.

The final network revealed several cell type related modules which matched with the cell type in benchmarking dataset as shown in FIG. 8C. The network formed clear immune cell type related modules. For instance, the upper-right corner represented the B cell and pre-B cell module, with CD78A and CD79B rated higher Pagerank (node size in FIG. 8C). Similarly, lower-right corner represented natural killer cell module, and middle-right region represented T cell as well as a transit from cytotoxic CD8 T cell to natural killer cell. The results demonstrated that, after implementing noise regularization, scRNA-seq data can be used to reconstruct gene-gene co-expression networks that better reflect the networks existed in biology.

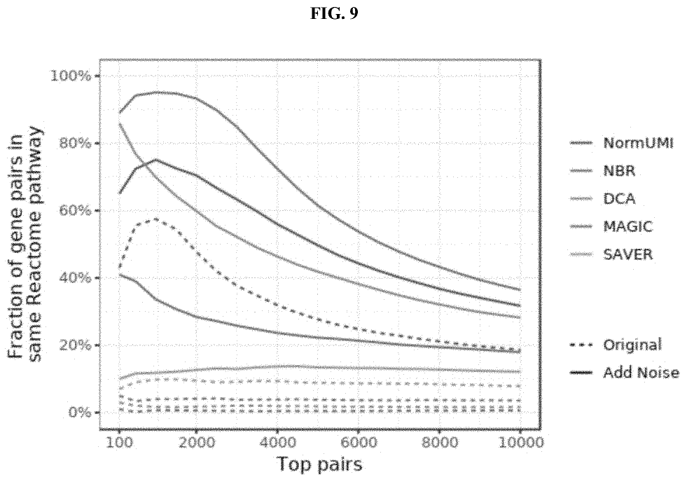

FIGS. 8A-8C show gene-gene correlation network inferred from scRNA-seq data. FIG. 8A and FIG. 8B show the comparison of Degree and Pagerank of each gene in the correlation networks constructed before and after applying noise regularization. Genes presented in one network, which were absent in the other networks, were assigned a zero value in the non-presenting network. Cell type marker genes, such as NKG7, CD79B, or HBB, had relative higher Degree and Pagerank after noise regularization. FIG. 8C shows network construction with refined gene-gene correlations. The scRNA-seq data were processed by applying NBR and noise regularization. Furthermore, the links which were not present in protein-protein interaction were removed. As shown in FIG. 8C, node size is proportional to a gene's Pagerank. Cell type marker genes, such as CD79A, CD79B, NKG7, GNLY, LYZ, or STMN1, have high Pagerank, indicating their importance in different cell types. Cell type related genes also formed cell type specific modules. FIG. 9 shows enrichment of top correlated gene pairs in Reactome pathways before and after applying noise regularization. X-axis indicates the top n gene pairs. Y-axis indicates the fraction of the n gene pairs appearing in the same pathway in Reactome database. Dashed lines and solid lines represent before and after noise regularization, respectively.

### Example 7. Determine the Optimal Noise Level

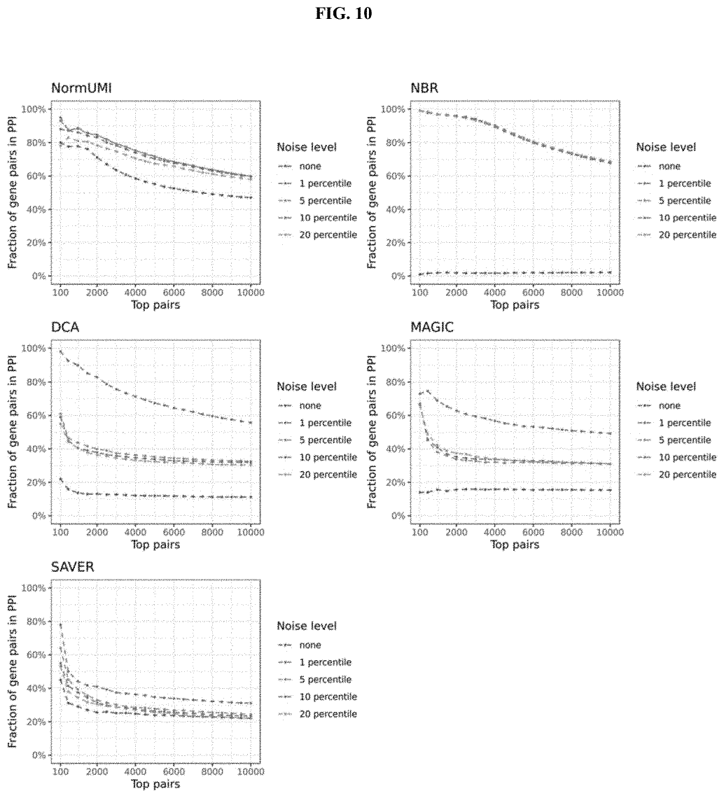

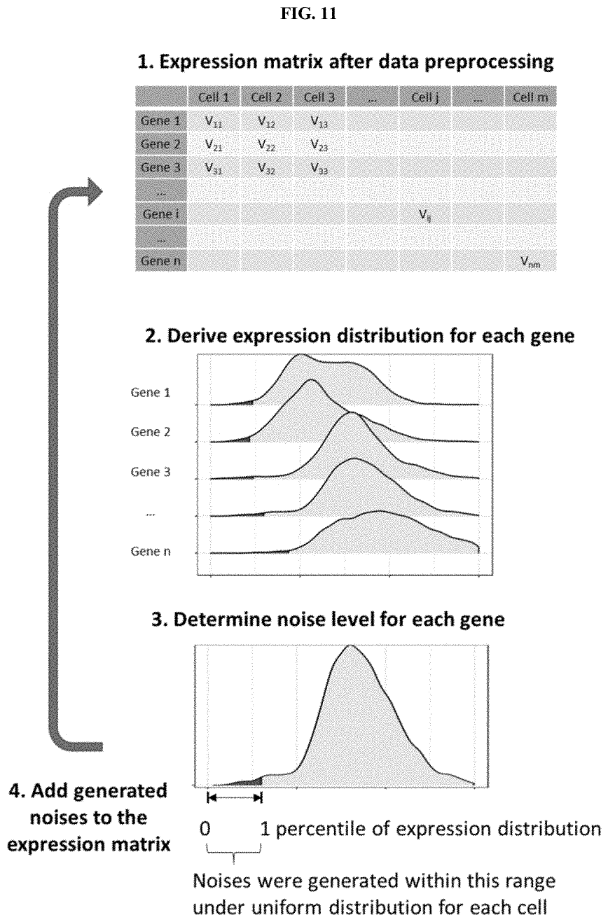

The optimal noise levels to be added during noise regularization were determined relative to the expression level of each gene. Different noise levels, such as 0.1, 1, 2, 5, 10, or 20 percentile of the expression level of each gene, were tested by applying five representative data preprocessing methods, e.g., NormUMI, NBR, DCA, MAGIC, and SAVER. The results indicate that 1 percentile optimally produced the highest protein-protein interaction enrichment across all five methods as shown in FIG. 10. Subsequently, random noises ranged from about 0 to 1 percentile of gene expression level were generated and added to the expression matrix as shown in FIG. 11. This noise regularization process significantly reduced the false correlations among the top gene pairs by generating more reliable gene-gene relationships.

As shown in FIG. 11, the noise regularization process included obtaining the expression matrix processed by a specific scRNA-seq preprocessing method, wherein this expression matrix contained n genes' expression in m cells. Assuming Vis the expression value of gene i in cell j, a random noise will be generated and added to V by the following procedures: (1) determine the expression distribution of gene i across all the cells; (2) take the 1st percentile from gene i's expression distribution as the maximal noise level for gene i, denoted as M (if M is smaller than a minimal value m, m will be used as the maximal noise level); (3) generate a random number ranging from 0 to M under uniform distribution; (4) add this random number to V to obtain the noise regularized expression value; and (5) repeat this procedure for every item in the expression matrix.

What is claimed is:

1. A method for improving data processing for gene-gene correlation, comprising:
processing gene expression data for normalization or imputation;
applying a noise regularization process to the normalized or imputed gene expression data; and
applying a gene-gene correlation calculation process to obtain correlated gene pairs.

2. The method of claim 1, wherein the gene expression data is single cell gene expression data.

3. The method of claim 1, wherein the noise regularization process comprises adding a random noise to an expression value of a gene in a cell in an expression matrix.

4. The method of claim 3, wherein the random noise is determined by an expression level of the gene.

5. The method of claim 3, wherein the random noise is determined by:
determining an expression distribution of the gene across all of the cells in the expression matrix;
taking from about 0.1 to about 20 percentile of an expression level of the gene as a maximal noise level;
generating a random number ranging from 0 to the maximal noise level under uniform distribution; and
adding the random number to the expression value of the gene in the cell in the expression matrix to obtain a noise regularized expression matrix.

6. The method of claim 3, wherein the random noise is determined by:
determining an expression distribution of the gene across all of the cells in the expression matrix;
taking one percentile of an expression level of the gene as a maximal noise level; x
generating a random number ranging from 0 to the maximal noise level under uniform distribution; and
adding the random number to the expression value of the gene in the cell in the expression matrix to obtain a noise regularized expression matrix.

7. The method of claim 1, wherein the gene-gene correlation calculation process is conducted within cell clusters.

8. The method of claim 1, further comprising enriching the gene expression data that is associated with the correlated gene pairs.

9. The method of claim 1, wherein Total Unique Molecular Identifier Normalization (NormUMI), Regularized Negative Binomial Regression (NBR), a deep count autoencoder network (DCA), Markov affinity-based graph imputation of cells (MAGIC), or single-cell analysis via expression recovery (SAVER) is used for processing gene expression data for normalization or imputation.

10. The method of claim 1, further comprising constructing a gene-gene correlation network based on the correlated gene pairs.

11. The method of claim 10, wherein the gene-gene correlation networks are cell type-specific.

12. The method of claim 10, further comprising using the gene-gene correlation networks for mapping molecular interactions, guiding experimental designs to investigate the biological events, discovering biomarkers, guiding comparative network analysis, guiding drug designs, identifying changes of gene-gene interactions by comparing healthy and disease states of cells, guiding drug development, predicting transcription regulation of genes, improving drug efficiency or identifying drug resistance factors.

13. A gene-gene correlation network, wherein the network is constructed based on correlated gene pairs, and wherein the correlated gene pairs are obtained using the method of claim 1.

14. A computer-implemented method for data processing for gene-gene correlation, comprising:
retrieving gene expression data;
processing the gene expression data for normalization or imputation;
applying a noise regularization process to the normalized or imputed gene expression data;
applying a gene-gene correlation calculation process to obtain correlated gene pairs, and
constructing a gene-gene correlation network based on the correlated gene pairs.

15. The method of claim 14, wherein the gene expression data is single cell gene expression data.

16. The method of claim 14, wherein the noise regularization process comprises adding a random noise to an expression value of a gene in a cell in an expression matrix.

17. The method of claim 16, wherein the random noise is determined by an expression level of the gene.

18. The method of claim 16, wherein the random noise is determined by:
determining an expression distribution of the gene across all of the cells in the expression matrix;
taking from about 0.1 to about 20 percentile of an expression level of the gene as a maximal noise level;
generating a random number ranging from 0 to the maximal noise level under uniform distribution; and
adding the random number to the expression value of the gene in the cell in the expression matrix to obtain a noise regularized expression matrix.

19. The method of claim 16, wherein the random noise is determined by:
determining an expression distribution of the gene across all of the cells in the expression matrix;
taking one percentile of an expression level of the gene as a maximal noise level;
generating a random number ranging from 0 to the maximal noise level under uniform distribution; and
adding the random number to the expression value of the gene in the cell in the expression matrix to obtain a noise regularized expression matrix.

20. The method of claim 14, wherein the gene-gene correlation calculation process is conducted within cell clusters.

21. The method of claim 14, further comprising enriching the gene expression data that is associated with the correlated gene pairs.

22. The method of claim 14, wherein Total Unique Molecular Identifier Normalization (NormUMI), Regularized Negative Binomial Regression (NBR), a deep count autoencoder network (DCA), Markov affinity-based graph imputation of cells (MAGIC), or single-cell analysis via expression recovery (SAVER) is used for processing gene expression data for normalization or imputation.

23. The method of claim 14, wherein the gene-gene correlation networks are cell type-specific.

24. The method of claim 14, further comprising using the gene-gene correlation networks for mapping molecular interactions, guiding experimental designs to investigate the biological events, discovering biomarkers, guiding comparative network analysis, guiding drug designs, identifying changes of gene-gene interactions by comparing healthy and disease states of cells, guiding drug development, predicting transcription regulation of genes, improving drug efficiency or identifying drug resistance factors.

25. A system for generating a gene-gene network, comprising:
a database configured to store gene expression data;
a memory configured to store instructions;
at least one processor coupled to the memory, wherein the at least one processor is configured to execute instructions for:
retrieving the gene expression data,
processing the gene expression data for normalization or imputation,
applying a noise regularization process to the normalized or imputed gene expression data,
applying a gene-gene correlation calculation process to obtain correlated gene pairs; and
constructing a gene-gene correlation network based on the correlated gene pairs; and
a user interface coupled to the processor and capable of receiving a query for gene-gene correlation and displaying the results of the correlated gene pairs and the constructed gene-gene correlation networks.

26. The system of claim 25, wherein the gene expression data is single cell gene expression data.

27. The system of claim 25, wherein the noise regularization process comprises adding a random noise to an expression value of a gene in a cell in an expression matrix.

28. The system of claim 27, wherein the random noise is determined by an expression level of the gene.

29. The system of claim 27, wherein the random noise is determined by:
determining an expression distribution of the gene across all of the cells in the expression matrix;
taking from about 0.1 to about 20 percentile of an expression level of the gene as a maximal noise level;
generating a random number ranging from 0 to the maximal noise level under uniform distribution; and
adding the random number to the expression value of the gene in the cell in the expression matrix to obtain a noise regularized expression matrix.

30. The system of claim 27, wherein the random noise is determined by:
determining an expression distribution of the gene across all of the cells in the expression matrix;
taking one percentile of an expression level of the gene as a maximal noise level;
generating a random number ranging from 0 to the maximal noise level under uniform distribution; and
adding the random number to the expression value of the gene in the cell in the expression matrix to obtain a noise regularized expression matrix.

31. The system of claim 25, wherein the gene-gene correlation calculation process is conducted with cell clusters.

32. The system of claim 25, wherein the at least one processor is further configured to enrich the gene expression data that is associated with the correlated gene pairs.

33. The system of claim 25, wherein Total Unique Molecular Identifier Normalization (NormUMI), Regularized Negative Binomial Regression (NBR), a deep count autoencoder network (DCA), Markov affinity-based graph imputation of cells (MAGIC), or single-cell analysis via expression recovery (SAVER) is used for processing gene expression data for normalization or imputation.

34. The system of claim 25, wherein the gene-gene correlation networks are cell type-specific.

35. The system of claim 25, wherein the at least one processor is further configured to utilize the gene-gene correlation networks for mapping molecular interactions, guiding experimental designs to investigate the biological events, discovering biomarkers, guiding comparative network analysis, guiding drug designs, identifying changes of gene-gene interactions by comparing healthy and disease states of cells, guiding drug development, predicting transcription regulation of genes, improving drug efficiency or identifying drug resistance factors.
